(19) United States
(12) Patent Application Publication
(10) Pub. No.: US 2006/0024335 A1
(43) Pub. Date: Feb. 2, 2006

(54) Oral compositions which mask the bitter taste of a bitter-tasting agent

(76) Inventor: Stier E. Roger, Clifton, NJ (US)

Correspondence Address: Richard M. Rosati, Esq.;KENYON & KENYON, One Broadway, New York, NY, 10004, US

(21) Appl. No.: 10/902,352

(22) Filed: Jul. 29, 2004

(51) Int. Cl. A61K 9/00

(52) U.S. Cl. 424/400

(57) ABSTRACT

An oral composition containing a bitter-tasting agent, such as for example, dextromethorphan, chlorhexidine, guaifenesin, pseudoephedrine, caffeine, peroxide, atorvastatin, aspirin, acetaminophen, diphenhydramine, doxylamine, sildenafil citrate, or loperamide, wherein the bitter taste ordinarily imparted by the bitter-tasting agent is abated or eliminated (i.e., masked) by an effective amount of a taste-receptor blocker, a taste-receptor competitor, a sweetener, and optionally, a flavor agent. Preferably, the taste-receptor blocker includes a hydrogenated ethoxylated glycerol ester, the taste-receptor competitor includes sodium citrate, and the sweetener includes sucralose and mono-ammonium glycyrrhizinate.

### FIELD OF THE INVENTION

The invention relates to oral compositions which ordinarily impart a bitter taste to the mouth. More particularly, the invention relates to oral compositions which contain a bitter-tasting agent, including but not limited to, dextromethorphan, chlorhexidine, guaifenesin, and pseudoephedrine, wherein the bitter taste of such agents is abated or eliminated.

### BACKGROUND OF THE INVENTION

There are many oral compositions known in the art which contain certain active ingredients which impart a bitter taste to the user. Many of these bitter-tasting agents are pharmaceuticals which are found in liquid compositions (such as solutions and syrups), solid compositions (such as capsules and tablets), and more recently, dissolvable films. For example, some of the more common bitter-tasting pharmaceutical agents include the cough suppressant dextromethorphan, the antimicrobial drug chlorhexidine, the expectorant guaifenesin, the decongestant pseudoephedrine, and the cholesterol-lowering drug atorvastatin. Thus, there are several types of oral compositions which are widely used and which impart a bitter taste to the user due to the bitter-tasting agents contained therein.

In regard to liquid compositions, there have been several attempts to develop additives to the liquid such that the otherwise present bitter taste will be lessened or masked. For example, U.S. Pat. No. 5,962,461 (“Anaebonam et al.”) is directed to a pleasant-tasting aqueous liquid composition of a bitter-tasting drug. Anaebonam et al. attempt to address the problem of the bitter taste by providing for an aqueous medium that is free of ethanol into which the bitter-tasting drug is dissolved or dispersed. See U.S. Pat. No. 5,962,461, at col. 1, lines 55-59; abstract. Specifically, the aqueous medium disclosed in Anaebonam et al. “consists essentially of water, about 5 to about 30 weight percent polyvinylpyrrolidone (PVP), about 35 to about 55 weight percent of a C₃-C₆ polyol, about 0.01 to about 0.5 weight percent ammonium glycyrrhizinate and one or more flavorants.” Id. at col. 1, lines 59-64. According to Anaebonam et al., the resulting liquid composition has a pleasant taste which is free from bitterness. See id. at col. 1, lines 64-67; abstract.

U.S. Pat. No. 5,633,006 (“Catania et al.”) is directed to a taste-masking composition of bitter pharmaceutical agents. The taste-masking composition disclosed in Catania et al. consists of the bitter pharmaceutical agent, a taste-masking component and a pharmaceutically acceptable carrier or diluent. See U.S. Pat. No. 5,633,006, at col. 3, lines 24-33; abstract. Furthermore, the taste-masking component disclosed by Catania et al. “is an alkaline earth oxide, an alkaline earth hydroxide or an alkaline hydroxide and does not interfere with the activity of the pharmaceutical agent.” Id. at abstract. According to the examples disclosed in Catania et al., the taste-masking composition may take the form of a solid chewable tablet, as well as a liquid suspension.

In addition, a relatively new type of oral composition for the delivery of active ingredients is a dissolvable film. Dissolvable films employ a water-soluble film-forming polymer in a water-oil emulsion containing active ingredients, and possibly flavors and sweeteners, in a film that is extruded and heated to drive off excess water. The finished sheets of film are then cut into strips, which are sized to deliver the correct amount of active ingredients. For example, U.S. Patent Application Publication No. 2003/0206941 to Leung et al. dislcoses fast dissolving orally consumable films and methods of producing such films, and is incorporated herein by reference in its entirety.

This relatively new dosing mechanism of dissolvable films has been adapted to the pharmaceutical industry as an alternative method for delivering medication to patients who are unable to use the more traditional methods of dosing (i.e., tablets or pills). By adjusting different levels of the polymers and other ingredients within the matrix of the films, release of the active ingredients can be controlled. With the introduction of dissolvable films in the pharmaceutical industry, masking of the unpleasant taste of numerous active ingredients has become essential as almost all pharmaceutical active ingredients are extremely bitter and cannot be tolerated by patients, which is why they are normally formed into coated tablets that are released in the stomach or intestines thereby eliminating the bitter experience. However, with the advance of dissolvable films, the active ingredients are now experienced within the oral cavity and need to be rendered more tasteful and user-friendly.

According to the present invention, an alternative solution to the problem of bitter-tasting oral compositions is proposed which is both highly effective and applicable to a wide range of oral compositions.

### SUMMARY OF THE INVENTION

It is an object of the invention to provide an oral composition containing a bitter-tasting agent, wherein the bitter taste ordinarily imparted by the bitter-tasting agent is masked.

It is a further object of the invention to provide such an oral composition wherein the masking agents do not themselves impart a bitter taste to the oral composition.

It is another object of the invention to provide a method for masking the bitter taste of an oral composition containing a bitter-tasting agent.

These and other objects of the invention which will become apparent from the following detailed description are achieved by providing an oral composition containing a bitter-tasting agent, wherein the bitter taste ordinarily imparted by the bitter-tasting agent is masked by effective amounts of a taste-receptor blocker, a taste-receptor competitor, a sweetener, and optionally, a flavor agent.

The taste-receptor blocker is any substance which coats the taste receptors in the mouth and thereby impedes or blocks direct contact between the taste receptors and the bitter-tasting agent contained in the oral composition. A preferred taste-receptor blocker is a hydrogenated ethoxylated glycerol ester.

The taste-receptor competitor is any substance which competes with the bitter-tasting agent at the ion channels in the taste buds to diminish or lessen the firings of the bitter receptors of the tongue. A preferred taste-receptor competitor is sodium citrate.

The sweetener is any substance which imparts a sweet taste in the mouth. A preferred sweetener for the oral composition of the present invention employs a combination of an initial sweetener, such as sucralose, and a delayed sweetener, such as mono-ammonium glycyrrhizinate.

In accordance with the invention, in an oral composition containing a bitter-tasting agent, the use of a taste-receptor blocker, a taste-receptor competitor, a sweetener, and optionally, a flavor agent, will serve to abate or eliminate the customary bitter taste attributable to the bitter-tasting agent. The resulting oral composition is therefore less bitter-tasting than would otherwise be expected from an oral composition containing a bitter-tasting agent.

### BRIEF DESCRIPTION OF THE DRAWINGS

FIG. 1 shows taste testing results for bitterness comparing an ordinary cough syrup containing a bitter-tasting agent (Example 1) to cough syrups of the invention according to Examples 2, 3 and 4.

FIG. 2 shows taste testing results for bitterness comparing a dissolvable film containing a bitter-tasting agent (Example 5) to a dissolvable film of the invention according to Example 6.

FIG. 3 shows taste testing results for bitterness comparing a dissolvable film containing a bitter-tasting agent (Example 7) to a dissolvable film of the invention according to Example 8.

### DETAILED DESCRIPTION OF THE INVENTION

In accordance with an embodiment of the invention, an oral composition containing a bitter-tasting agent is treated with effective amounts of a taste-receptor blocker, a taste-receptor competitor, and a sweetener such that the bitter taste typically imparted by the bitter-tasting agent is effectively masked. That is, this combination of three components, a taste-receptor blocker, a taste-receptor competitor, and a sweetener, serves to abate or eliminate the customary bitter taste attributable to the bitter-tasting agent, resulting in a more pleasant-tasting oral composition. In addition, a flavor agent may also be added to the oral composition in an amount which is effective to abate or eliminate the bitter taste ordinarily imparted by the bitter-tasting agent, when combined with the taste-receptor blocker, the taste-receptor competitor, and the sweetener.

Although not wishing to be bound by any theory, it is believed that the combination of these specific components of the oral composition (i.e., the taste-receptor blocker, the taste-receptor competitor, the sweetener, and optionally, the flavor agent) serves to mask the bitter taste ordinarily imparted by the bitter-tasting agent as follows.

During the experience of “tasting,” several physiological and psychological events occur simultaneously. Anatomically, taste cells reside within specialized structures called taste buds, which are located on the tongue and soft palate. The majority of taste buds are located within papillae, which are the tiny projections on the surface of the tongue that give it its velvety appearance. Taste buds are onion-shaped structures of between 50 and 100 taste cells, each of which possesses finger-like projections called microvilli that protrude through an opening at the top of the taste bud called the taste pore. Chemicals from food (called “tastants”) dissolve in saliva and contact the taste cells via the taste pore. There they either interact with surface proteins of the cells called taste receptors, or they interact with pore-like proteins called ion channels. These interactions cause electrical changes within the taste cells that trigger them to send chemical signals that translate into neurotransmission to the brain. Salt and sour tastes are of the ion channel type of responses, while sweet and bitter tastes are surface protein responses. The electrical responses that send the signal to the brain are a result of a varying concentration of charged atoms or ions within the taste cell. These cells normally have a net negative charge. Tastants alter this state by using varying means to increase the concentration of positive ions within the taste cell. This depolarization causes the taste cells to release neurotransmitters, prompting neurons connected to the taste cells to relay electrical messages to the brain. In the case of a bitter taste, such as for example quinine, stimuli act by binding to G-protein coupled receptors on the surface of the taste cell. This then prompts the protein subunits of alpha, beta, and gamma to split and activate a nearby enzyme. This enzyme then converts a precursor within the cell into a “second messenger”. The second messenger causes the release of calcium ions (Ca⁺²) from the endoplasmic reticulum of the taste cell. The resulting build-up of calcium ions within the cell leads to depolarization and neurotransmitter release. The signal now sent to the brain is interpreted as a bitter taste.

Physiologically and bio-chemically, the nature of the ion channel response (pore-like proteins) for salty and sour tastes is quite different from the taste receptor response (surface proteins) for sweet and bitter tastes. The salt taste occurs via the ion channel response, and is the response to, for example, a salt such as sodium chloride (Na⁺ and Cl⁻). The sodium ions (Na⁺) enter the receptor cells via the sodium ion channels (amiloride-sensitive sodium channels). The entry of the sodium ions causes a depolarization, calcium ions (Ca⁺²) enter through voltage-sensitive calcium channels, and transmitter release occurs and results in increased firing in the primary afferent nerve. On the other hand, the sour taste is the response to acid, and acid is characterized by excess protons (H+). Protons block the potassium (K⁺) channels, which are responsible for maintaining the cell membrane potential at a hyperpolarized level (close to the K⁺ equilibrium potential of −85 mV). Just as described above, the blocking of these channels causes depolarization within the cell, Ca⁺2 entry, transmitter release and increased firing in the primary afferent nerve. While the salt and sour taste sensations employ different channels to enter and affect the taste cells, the end result of each is very similar. Compounds eliciting a salt or sour taste are less diverse than those eliciting a sweet taste, and they are typically ions.

Generally speaking, one class of stimuli will be most effective in eliciting the highest frequency discharge because receptor specificity is considered relative as opposed to an all-or-none response. In other words, the differences between stimuli are not so much a difference between firing and non-firing of the neurons, but is in fact the differences in the amount of firing of the neurons. This consideration would explain, for example, why a sweet compound might reduce the perception of a bitter compound. That is, both interact with the taste receptors, thereby leading to the neural firing for each taste. The overall taste perception of the brain will then be dependent upon the amount of firing of the receptors. By causing the receptors of sweetness to become engaged while the bitterness receptors are engaged, for example, reduces the net effect of both taste sensations to the brain. That is, it would seem that a method of diminishing the overall response to one stimulus would be to introduce additional stimuli.

Again, although not wishing to be bound by any particular theory, it is no longer believed that there exists a “tongue map” for taste buds with each area of the tongue perceiving only certain sensations; instead, it is believed that taste cells respond to all sensations in different ways. The brain understands the experience of “taste” not as fired neurons or non-fired neurons, but as the amount of firing of the neurons. If a bitter-tasting agent is present in sufficient concentration, the neurons will be firing rapidly from the bitter receptors of all cells. However, the introduction of a salt or sour taste (via a taste-receptor competitor) and a sweet taste (via a sweetener), in addition to the bitter-tasting agent complicates the overall neuron response, as neural responses to each taste (bitter, salt and/or sour, and sweet) produce responses in competition with each other. Thus, the strong taste of a bitter-tasting agent within an oral composition (such as dextromethorphan present as a cough suppressant) now becomes less intense in the presence of a taste-receptor competitor and a sweetener.

Thus, it is believed that the foregoing discussion explains how the combination(s) of the bitter-masking components (the taste-receptor competitor, the taste-receptor blocker and the sweetener) of the composition of the invention effectively masks the customary bitter taste attributable to a bitter-tasting agent. That is, while the taste-receptor blocker coats the taste receptors within the taste buds and thereby impedes or blocks direct contact between the taste receptors and the bitter-tasting agent, the taste-receptor competitor competes with the bitter-tasting agent at the ion channels in the taste buds, and the sweetener competes with the bitter-tasting agent for the remaining, available taste receptors within the taste buds.

The bitter-tasting agent of the oral composition of the invention is any agent which ordinarily imparts a bitter taste to the oral composition. For example, such bitter-tasting agents include, but are not limited to: dextromethorphan; chlorhexidine; guaifenesin; pseudoephedrine; caffeine; peroxide; atorvastatin; aspirin; acetaminophen; diphenhydramine; doxylamine; sildenafil citrate; and loperamide. In addition, as would be understood by one of ordinary skill in the art, the amount of the bitter-tasting agent present in the oral composition of the invention will vary depending upon the particular bitter-tasting agent employed in the oral composition, and all such amounts of the bitter-tasting agent are within the scope of the present invention.

As described above, the taste-receptor competitor which is is used in the oral composition of the invention is any substance which competes with the bitter-tasting agent at the ion channels in the taste buds to diminish or lessen the firings of the bitter receptors of the tongue. That is, as the taste receptors respond to the taste-receptor competitor, the response to the bitter-tasting agent is reduced, thereby causing an overall reduction of the bitter taste. Thus, the taste-receptor competitor generally includes those substances which ordinarily impart a salt or sour taste. Examples of suitable taste-receptor competitors include, but are not limited to: citric acid, sodium salts of citric acid (sodium citrates), and calcium salts of citric acid (calcium citrates); phosphoric acid, sodium salts of phosphoric acid (sodium phosphates), and monobasic calcium salts of phosphoric acid; sodium chlorides; and hydroxy acids which include glycolic, lactic, hydroxybutyric, mandeliec, glycergic, malic, tartaric, and mesotartaric acids, and salts of such hydroxy acids (such salts including sodium and calcium as well as for tartaric acid, dipotassium, dissodium, and diammonium). Preferably, the taste-receptor competitor is or includes sodium citrate.

The amount of taste-receptor competitor to be included in the oral composition of the invention is any amount which is effective to mask the customary bitter taste attributable to the bitter-tasting agent, when combined with effective amounts of the taste-receptor blocker and the sweetener, and optionally, the flavor agent. For example, when the taste-receptor competitor comprises sodium citrate, the amount of sodium citrate to be included in the oral composition is preferably from about 0.25% to about 2.0% of the total weight of the oral composition, and most preferably about 1.0% of the total weight of the oral composition.

As also described above, the taste-receptor blocker which is used in the oral composition of the invention is any substance which coats the taste receptors in the mouth and thereby impedes or blocks direct contact between the taste receptors and the bitter-tasting agent contained in the oral composition. Thus, the bitter taste ordinarily imparted by the bitter-tasting agent in the oral composition is thereby abated or eliminated.

A particularly effective class of compounds which can function as taste-receptor blockers for a bitter-tasting agent are hydrogenated, ethoxylated glycerol esters. These types of compounds are commercially available and may be formed in a well-known manner, namely by the ethoxylation of glycerol. The ethoxylation may be accomplished by reacting the glycerol with ethylene oxide such that hydrogen bonding to the oxygen makes the polyethylene end of the molecule more soluble. As the ethoxylation number decreases, the fat character of the molecule and hence its efficacy in coating and blocking the taste receptors increases, but the solubility usually decreases thereby decreasing clarity. If the fat characteristics of the compound are too great, solubility in the oral composition is adversely affected which results in an undesirable cloudiness for the product. Good solubility is essential for product clarity. Accordingly, the taste-receptor blocker should preferably be selected so as to strike the proper balance between coating efficacy on the one hand and clarity on the other.

The hydrogenated ethoxylated glycerol esters can be prepared by hydrogenating castor oil and treating the resulting product with from about 10 to 200 moles of ethylene glycol. The ethoxylated compounds are designated as PEG (numeral) hydrogenated castor oil in accordance with the dictionary of the Cosmetics, Toiletries and Fragrance Association, 3^{rd} Ed. wherein the numeral following PEG indicates the degree of ethoxylation, i.e. the number of moles of ethylene oxide added. Ethoxylation numbers in the range of from 35 to 60 have been found to provide the best results in terms of good solubility and good clarity (i.e., minimal or no cloudiness). One commercially available compound which works particularly well is sold by the BASF Company under the trade name CREMOPHOR®. This compound is a hydrogenated ethoxylated castor oil. It has been found that CREMOPHOR® 35 through CREMOPHOR®60 (particularly CREMOPHOR® 40) work particularly well in accordance with the present invention.

The amount of taste-receptor blocker to be included in the oral composition of the invention is any amount which is effective to mask the customary bitter taste attributable to the bitter-tasting agent, when combined with effective amounts of the taste-receptor competitor and the sweetener, and optionally, the flavor agent. The amount of taste-receptor blocker incorporated in the oral composition of the invention will depend upon the amount of bitter-tasting agent contained in the oral composition as well as the degree to which the bitter taste imparted by the bitter-tasting agent is desired to be reduced. For example, when the taste-receptor blocker comprises CREMOPHOR® 40, the amount of CREMOPHOR® 40 to be included in the oral composition is preferably from about 2.0% to about 4.0% of the total weight of the oral composition.

The sweetener which is used in the oral composition of the invention is any substance which imparts a sweet taste in the mouth. There are a variety of high-intensity sweeteners that can be used either alone or in combination with each other to provide a specific sweetness profile. The sweetener may include an initial sweetener that provides an initial, intense sweetness which declines rapidly with time, and/or a delayed sweetener that provides a less intense sweetness initially which builds in intensity over time to extend the sweetness profile. An exemplary delayed sweetener is mono-ammonium glycyrrhizinate (“MAG”), and examples of initial sweeteners include, but are not limited to: saccharin; sucralose; neotame; alitame; aspartame; cyclamate; thaumatin; dihydrochalcones; and acesulfame potassium (acesulfame K) compounds.

A preferred sweetener for the oral composition of the present invention employs a sweetness profile which is modified to accommodate the necessity for prolonged sweetness in the presence of a bitter-tasting agent. That is, the sweetness profile of an initial sweetener, such as sucralose, is initially intense with a marked decline thereafter, while the sweetness profile of a delayed sweetener, such as mono-ammonium glycyrrhizinate, is less intense initially but builds in intensity over time. Therefore, a preferred sweetener for the oral composition of the present invention is the combination of these two sweeteners which provides a sweetness profile that successfully responds to the bitter-tasting agent.

The amount of sweetener to be included in the oral composition of the invention is any amount which is effective to mask the customary bitter taste attributable to the bitter-tasting agent, when combined with effective amounts of the taste-receptor blocker, the taste-receptor competitor, and optionally, the flavor agent. For example, when the sweetener includes mono-ammonium glycyrrhizinate the amount of mono-ammonium glycyrrhizinate to be included in the oral composition is preferably from about 0.01% to about 1.0% of the total weight of the oral composition, and most preferably about 0.03% to about 0.3% of the total weight of the oral composition.

In addition, the oral composition of the invention may optionally contain a flavor agent. Any flavor agent or agents may be used in accordance with the invention, including those known to the skilled artisan, such as, natural and artificial flavors. These flavor agents may be chosen from synthetic flavor oils and flavoring aromatics, and/or oils, oleo resins and extracts derived from plants, leaves, flowers, fruits and so forth, and combinations thereof. Representative flavor oils include: cinnamon oil, peppermint oil, clove oil, bay oil, eucalyptus oil, thyme oil, cedar leaf oil, oil of nutmeg, oil of sage, and oil of bitter almonds. Also useful are artificial, natural or synthetic fruit flavors such as vanilla, and citrus oil, including lemon, orange, grape, lime and grapefruit and fruit essences including apple, pear, peach, strawberry, raspberry, cherry, plum, pineapple, apricot and so forth. Any of these flavor agents may be used individually or in admixture. Commonly used flavors include mints such as peppermint, menthol, artificial vanilla, cinnamon derivatives, and various fruit flavors, whether employed individually or in admixture. Flavor agents such as aldehydes and esters including cinnamyl acetate, cinnamaldehyde, citral, diethyllacetal, dihydrocarvyl acetate, eugenyl formate, p-methylanisole, and so forth may also be used. Generally, any flavoring or food additive such as those described in Chemicals Used in Food Processing, pub 1274 by the National Academy of Sciences, pages 63-258 may be used as flavor agents in the invention.

When present in the oral composition of the invention, the flavor agent is present in an amount which is effective to abate or eliminate the bitter taste ordinarily imparted by the bitter-tasting agent, when combined with effective amounts of the taste-receptor blocker, the taste-receptor competitor, and the sweetener. For example, when the oral composition is a liquid, the amount of the flavor agent to be included therein is preferably from about 0.5% to about 2.0% of the total weight of the oral composition, and when the oral composition is a dissolvable film, the amount of the flavor agent to be included therein is preferably from about 3.0% to about 15.0% of the total weight of the oral composition.

The oral composition of the invention may be in any of the forms known in the art, including but not limited to: a liquid pharmaceutical composition such as a solution, suspension, emulsion, or the like; a solid pharmaceutical composition such as a solid dosage formulation; a dissolvable film; a toothpaste; a mouthwash; a tooth powder; a chewing gum; a dental cream or gel; and a denture adhesive composition. Preferably, the oral composition of the invention is in the form of a dissolvable film, or a liquid pharmaceutical composition such as a cough syrup or solution.

In general, the oral compositions of this invention are prepared utilizing techniques well known to those of ordinary skill in the art. As such, the oral compositions of this invention may include various other components which are customarily used in the preparation of such oral compositions, and which would be known to those of skill in the art.

For example, when the oral composition of the invention is in the form of a liquid pharmaceutical composition, or even a toothpaste, dental cream or gel, such a form typically includes a liquid carrier material for the bitter-tasting agent and the active ingredient(s) which mask the bitter taste thereof. The carrier material may comprise water, typically in an amount of from about 10% to about 90% by weight of the oral composition. Carrier materials include, but are not limited to, polyethylene glycol (PEG), propylene glycol, glycerin or mixtures thereof. In addition, the oral composition may include humectants, such as, for example, sorbitol, glycerin, and polyalcohols. Particularly advantageous liquid ingredients comprise mixtures of water with polyethylene glycol or glycerin and sorbitol. A gelling agent (thickening agent) including natural or synthetic gums, such as sodium carboxymethylcellulose, hydroxyethyl cellulose, methyl cellulose and the like, may also be used, typically in the range of about 0.15% to about 1.30% by weight of the oral composition. In a toothpaste, dental cream or gel, the liquids and solids are proportioned to form a creamy or gelled mass which is extrudable from a pressurized container or from a collapsible tube.

The oral composition of this invention may also include a thickening agent or binder. For example, the thickening agent or binder may be selected from the group consisting of finely particulate gel silicas and nonionic hydrocolloids, such as carboxymethyl cellulose, sodium hydroxymethyl cellulose, hydroxyethyl cellulose, hydroxypropyl guar, hydroxyethyl starch, polyvinyl pyrrolidone, vegetable gums, such as tragacanth, agar agar, carrageenans, gum arabic, xanthan gum, guar gum, locust bean gum, carboxyvinyl polymers, fumed silica, silica clays and the like, and combinations thereof. For example, a preferred thickening agent for use in toothpastes is carrageenan available under the trade names GELCARIN® and VISCARIN® from FMC Biopolymers, Philadelphia, Pa., U.S.A. Other thickening agents or binders are polyvinyl pyrrolidone available from Noveon, Inc. Cleveland, Ohio, U.S.A. under the trademark CARBOPOL®, fumed silica under the trademark CAB-O-SIL® available from Cabot Corporation, Boston, Mass., U.S.A., and silica clays available from Laporte Industries, Ltd., London, U.K. under the trademark LAPOINTE®. The thickening agent or binder may be used with or without a carrier, such as glycerol, polyethylene glycol (e.g., PEG-400), or combinations thereof; however, when a carrier is used, preferably up to about 5% thickening agent or binder, more preferably from about 0.1% to about 1.0%, is combined with preferably from about 95.0% to about 99.9% carrier, more preferably from about 99.0% to about 99.9%, based on the total weight of the thickening agent/carrier combination. Furthermore, when the thickening agent or binder is a hydrated silica and it is used with a carrier, preferably from about 5% to about 10% thickening agent or binder is combined with preferably from about 90% to about 95% carrier, based on the total weight of the thickening agent/carrier combination.

The oral composition of the invention may also contain coloring agents or colorants, such as colors, dyes, pigments and particulate substances, in amounts effective to produce the desired color of the particular oral composition. The coloring agents (colorants) useful in the invention include the pigments such as titanium dioxide, which may be incorporated in amounts of up to about 2% by weight of the oral composition, and preferably less than about 1% by weight. Colorants may also include natural food colors and dyes suitable for food, drug and cosmetic applications. For example, food grade and/or pharmaceutically acceptable coloring agents, dyes, or colorants, as would be understood to one skilled in the art, include FD&C colorants such as primary FD&C Blue No. 1, FD&C Blue No. 2, FD&C Green No. 3, FD&C Yellow No. 5, FD&C Yellow No. 6, FD&C Red No. 3, FD&C Red No. 33 and FD&C Red No. 40 and lakes FD&C Blue No. 1, FD&C Blue No. 2, FD&C Yellow No. 5, FD&C Yellow No. 6, FD&C Red No. 2, FD&C Red No. 3, FD&C Red No. 33, FD&C Red No. 40 and combinations thereof.

In addition, the oral composition of the invention may also include a surfactant, such as sodium lauryl sulfate (SLS) (preferably in an amount of from about 1% to about 2% of the total weight of the oral composition), and/or a preservative, such as sodium benzoate (preferably in an amount of about 0.2% of the total weight of the oral composition).

### EXAMPLES

Specific preferred embodiments of the invention will now be described with reference to the following examples which should be regarded in an illustrative rather than a restrictive sense.

The hydrogenated ethoxylated glycerol ester used in any of the following Examples was CREMOPHOR® 40 (BASF). In addition, CREMOPHOR® 35 through CREMOPHOR® 60 can be used in accordance with the invention.

The mono-ammonium glycyrrhizinate used in any of the following Examples was MAGNASWEET® 100 (Mafco). MAGNASWEET® 120, 125, 130, 165 and 365 can also be used in accordance with the invention.

### Example 1

The oral composition of Example 1 was a cough syrup, prepared by techniques well-known to those of ordinary skill in the art. Specifically, the cough syrup of Example 1 was purchased as the cough syrup sold under the trade name ROBITUSSIN® CF. The cough syrup of Example 1 served as a control without the addition of any of the active agents of the invention which would mask the bitter taste of the bitter-tasting agents contained therein. Specifically, the bitter-tasting agents contained in the cough syrup of Example 1 were dextromethorphan HBr USP 10 mg, guaifenesin USP 100 mg, and pseudoephedrine HCl USP 30 mg (wherein the mg amounts refer to each 5 ml teaspoon).

### Example 2

In Example 2, the same cough syrup according to Example 1 was initially obtained. However, in Example 2, additional ingredients were added to the cough syrup in the following amounts:

Example 2

| | wt. % of oral | |
|---|---|---|
| Ingredient | composition | weight (grams) |
| Example 1 cough syrup | 96.97 | 969.70 |
| hydrogenated ethoxylated | 2.00 | 20.00 |
| glycerol ester | | |
| mono-ammonium | 0.03 | 0.30 |
| glycyrrhizinate | | |
| sodium citrate | 1.00 | 10.00 |

### Example 3

In Example 3, the same cough syrup according to Example 1 was initially obtained. However, in Example 3, additional ingredients were added to the cough syrup in the following amounts:

Example 3

| | wt. % of oral | |
|---|---|---|
| Ingredient | composition | weight (grams) |
| Example 1 cough syrup | 94.97 | 949.70 |
| hydrogenated ethoxylated | 4.00 | 40.00 |
| glycerol ester | | |
| mono-ammonium | 0.03 | 0.30 |
| glycyrrhizinate | | |
| sodium citrate | 1.00 | 10.00 |

### Example 4

In Example 4, the same cough syrup according to Example 1 was initially obtained. However, in Example 4, additional ingredients were added to the cough syrup in the following amounts:

Example 4

| | wt. % of oral | |
|---|---|---|
| Ingredient | composition | weight (grams) |
| Example 1 cough syrup | 93.97 | 939.70 |
| hydrogenated ethoxylated | 3.00 | 30.00 |
| glycerol ester | | |
| mono-ammonium | 0.03 | 0.30 |
| glycyrrhizinate | | |
| sodium citrate | 1.00 | 10.00 |
| flavor agent (cherry) | 2.00 | 20.00 |

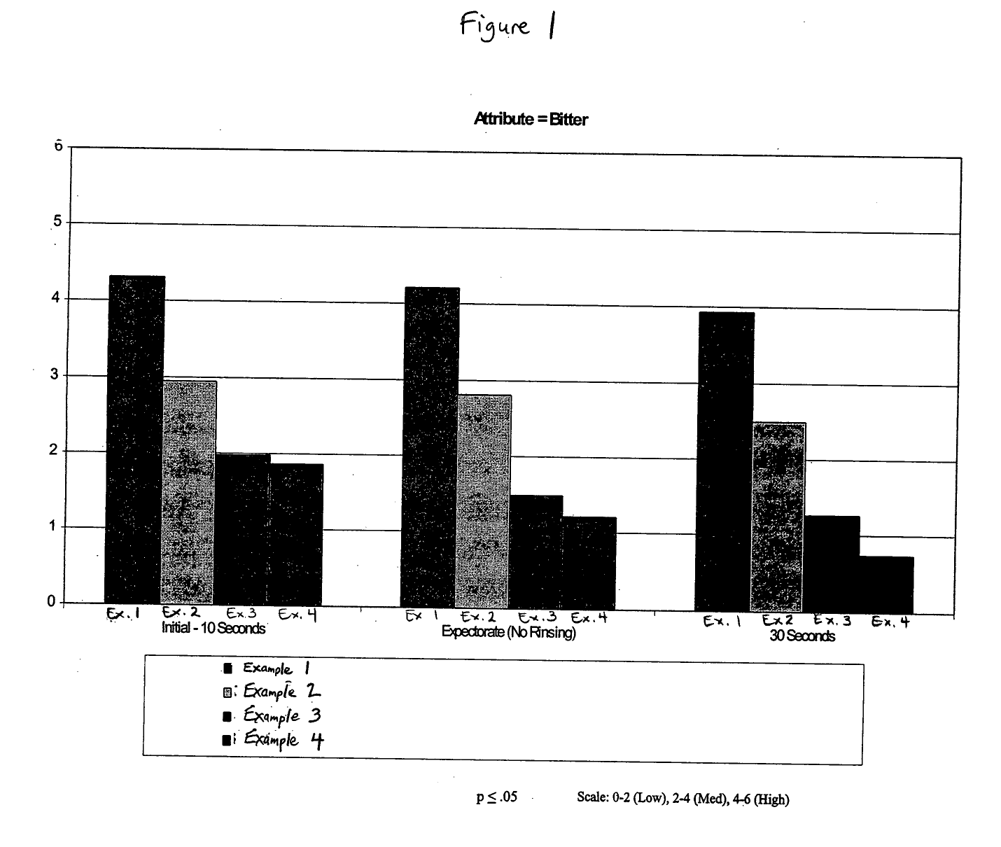

FIG. 1 shows taste testing results for bitterness from a six-member panel comparing the tastes of the cough syrups prepared according to Examples 1 through 4 in terms of an initial taste, an expectorate taste, and a taste after 30 seconds. As can be seen in FIG. 1, the bitter taste ordinarily imparted by the dextromethorphan HBr, guaifenesin, and pseudoephedrine HCl is prevalent in Example 1 in the initial taste, the expectorate taste, and the taste after 30 seconds. However, it can also be seen that in the cough syrups according to Examples 2 through 4, this bitter taste is progressively diminished in the initial taste, the expectorate taste, and the taste after 30 seconds, most notably in the taste after 30 seconds of the cough syrup according to Example 4.

### Example 5

The oral composition of Example 5 was in the form of a dissolvable film, which was prepared using materials and techniques well-known to those of ordinary skill in the art, such as those disclosed in U.S. Patent Application Publication No. 2003/0206941. The dissolvable film of Example 5 included, inter alia, hydroxypropylmethyl cellulose and hydroxypropyl cellulose as film-forming agents, a polyethylene oxide compound, and the extremely bitter-tasting active ingredient calcium atorvastatin, which is the active ingredient in the cholesterol-lowering medication sold under the trade name LIPITOR®. The dissolvable film of Example 5 served as a control without any of the active agents of the invention which would mask the bitter taste of the bitter-tasting agents (i.e., calcium atorvastatin) within the dissolvable film.

### Example 6

In Example 6, the same dissolvable film according to Example 5 was initially prepared. However, in Example 6, additional ingredients were added to the dissolvable film in the following amounts:

Example 6

| | wt. % of oral | |
|---|---|---|
| Ingredient | composition | weight (grams) |
| Example 5 dissolvable film | 79.10 | 791.00 |
| hydrogenated ethoxylated | 2.00 | 20.00 |
| glycerol ester | | |
| mono-ammonium | 0.30 | 3.00 |
| glycyrrhizinate | | |
| flavor agent (citrus mint | 10.00 | 100.00 |
| with menthol) | | |
| sodium chloride | 8.00 | 80.00 |
| sucralose | 0.60 | 6.00 |

Specifically, these additional ingredients of Example 6 as listed above were added as follows. A water phase was prepared wherein water, the sweeteners (i.e., mono-ammonium glycyrrhizinate and sucralose), the bitter-tasting agent (i.e., calcium atorvastatin), the taste-receptor competitor (i.e., sodium chloride), the film-forming agents, and the polyethylene oxide compound were combined. An oil phase was prepared wherein the taste-receptor blocker (i.e., hydrogenated ethoxylated glycerol ester) and the flavor agent were combined. Then, the water phase was added to the oil phase with high shear mixing (via a SILVERSON® mixer), and the combination was mixed until good emulsion was achieved.

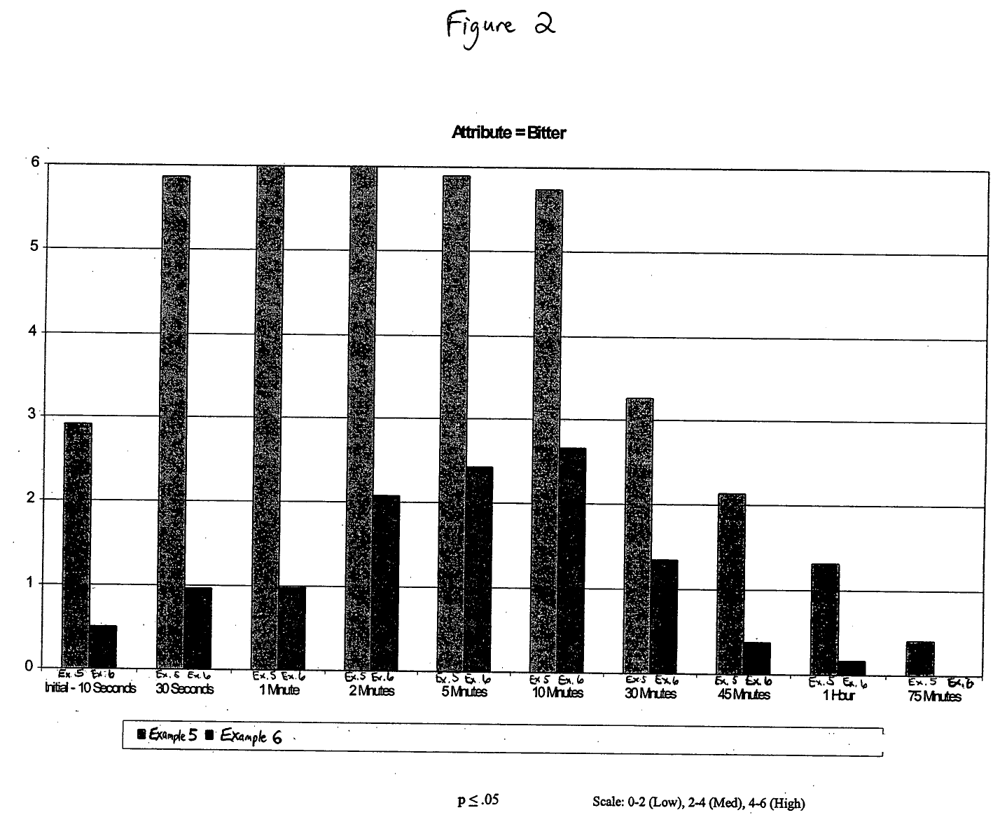

FIG. 2 shows taste testing results for bitterness from a six-member panel comparing the tastes of the dissolvable film prepared according to Examples 5 and 6 spanning a time frame from the taste after 10 seconds (an initial taste) to the taste after 75 minutes. As can be seen in FIG. 2, the bitter taste ordinarily imparted by the calcium atorvastatin is greatly diminished at each and every point of the aforementioned time frame in the dissolvable film of the invention according to Example 6.

### Example 7

The oral composition of Example 7 was in the form of a dissolvable film, which was prepared using materials and techniques well-known to those of ordinary skill in the art, such as those disclosed in U.S. Patent Application Publication No. 2003/0206941. The dissolvable film of Example 7 included, inter alia, hydroxypropylmethyl cellulose and hydroxypropyl cellulose as film-forming agents, a polyethylene oxide compound, and the bitter-tasting agent caffeine, which was present at a 15 mg dose (comprising 19% by weight of the dissolvable film). The dissolvable film of Example 7 served as a control without any of the active agents of the invention which would mask the bitter taste of the bitter-tasting agent (i.e., caffeine) within the dissolvable film.

### Example 8

In Example 8, the same dissolvable film according to Example 7 was initially prepared. However, in Example 8, additional ingredients were added to the dissolvable film in the following amounts:

Example 8

| | wt. % of oral | |
|---|---|---|
| Ingredient | composition | weight (grams) |
| Example 7 oral composition | 84.42 | 844.20 |
| hydrogenated ethoxylated | 2.00 | 20.00 |
| glycerol ester | | |
| flavor agent (coffee) | 11.00 | 110.00 |
| sucralose | 2.50 | 25.00 |
| citric acid | 0.08 | 0.80 |

Specifically, these additional ingredients of Example 8 as listed above were added as follows. A water phase was prepared wherein water, the sweetener (i.e., sucralose), the bitter-tasting agent (i.e., caffeine), the taste-receptor competitor (i.e., citric acid), the film-forming agents, and the polyethylene oxide compound were combined. An oil phase was prepared wherein the taste-receptor blocker (i.e., hydrogenated ethoxylated glycerol ester) and the flavor agent were combined. Then, the water phase was added to the oil phase with high shear mixing (via a SILVERSON® mixer), and the combination was mixed until good emulsion was achieved.

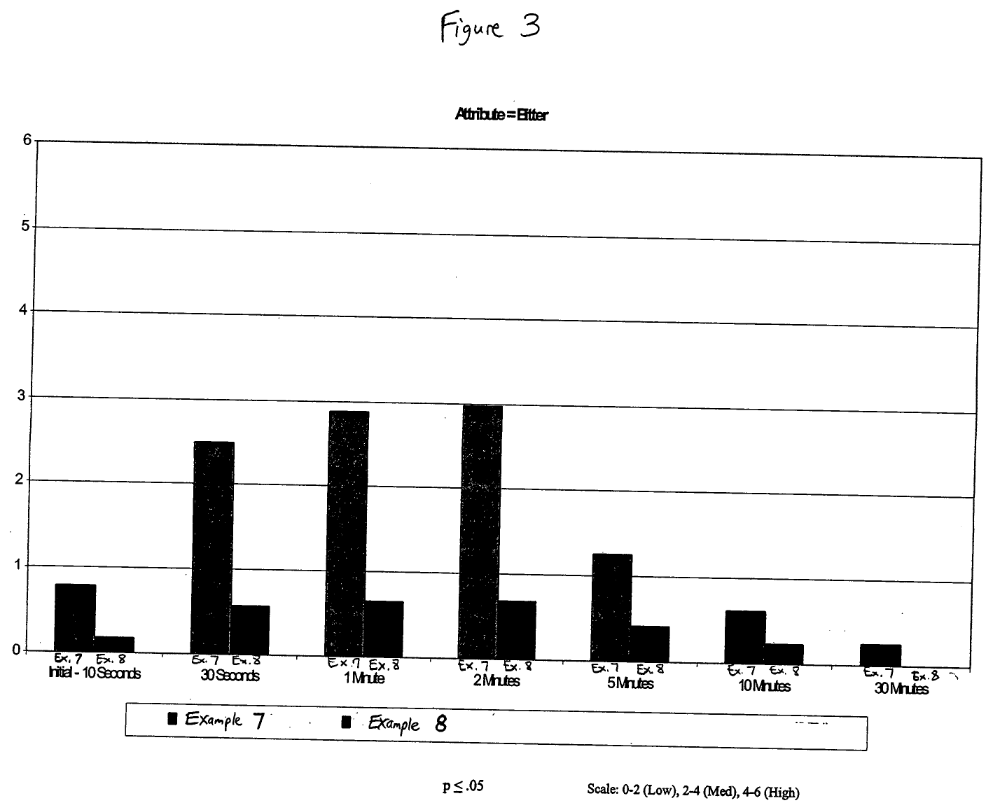

FIG. 3 shows taste testing results for bitterness from a six-member panel comparing the tastes of the dissolvable films prepared according to Examples 7 and 8 spanning a time frame from the taste after 10 seconds (an initial taste) to the taste after 30 minutes. As can be seen in FIG. 3, the bitter taste ordinarily imparted by the caffeine is greatly diminished at each and every point of the aforementioned time frame in the dissolvable film of the invention according to Example 8.

As demonstrated by the test results of Examples 1 through 8 and the results depicted in FIGS. 1 through 3, the bitter taste of an oral composition ordinarily imparted by the bitter-tasting agent(s) contained therein is effectively masked by the addition of the combination of a taste-receptor blocker (e.g., hydrogenated ethoxylated glycerol ester), a taste-receptor competitor (e.g., sodium citrate, sodium chloride, or citric acid) and a sweetener (e.g., mono-ammonium glycyrrhizinate and/or sucralose), and optionally, a flavor agent.

What is claimed is:

1. An oral composition comprising a bitter-tasting agent, a taste-receptor blocker, a taste-receptor competitor, and a sweetener, wherein the taste-receptor blocker, the taste-receptor competitor and the sweetener are present in amounts which are effective to abate or eliminate the bitter taste ordinarily imparted by the bitter-tasting agent.

2. The oral composition of claim 1, wherein the bitter-tasting agent is selected from the group consisting of dextromethorphan, chlorhexidine, guaifenesin, pseudoephedrine, caffeine, peroxide, atorvastatin, aspirin, acetaminophen, diphenhydramine, doxylamine, sildenafil citrate, loperamide, and combinations thereof.

3. The oral composition of claim 1, wherein the taste-receptor blocker is selected from the group consisting of hydrogenated ethoxylated glycerol esters and combinations thereof.

4. The oral composition of claim 1, wherein the taste-receptor blocker includes a hydrogenated ethoxylated glycerol ester having an ethoxylation number in the range of from 35 to 60.

5. The oral composition of claim 1, wherein the taste-receptor blocker includes a hydrogenated ethoxylated glycerol ester having an ethoxylation number of 40.

6. The oral composition of claim 5, wherein the hydrogenated ethoxylated glycerol ester having an ethoxylation number of 40 is present in an amount of from about 2.0% to about 4.0% of the total weight of the oral composition.

7. The oral composition of claim 1, wherein the sweetener is selected from the group consisting of mono-ammonium glycyrrhizinate, sucralose and combinations thereof.

8. The oral composition of claim 1, wherein the taste-receptor competitor is selected from the group consisting of citric acid, sodium salts of citric acid, calcium salts of citric acid, phosphoric acid, sodium salts of phosphoric acid, monobasic calcium salts of phosphoric acid, sodium chloride, hydroxy acids, salts of hydroxy acids, and combinations thereof.

9. The oral composition of claim 1, wherein the taste-receptor competitor is selected from the group consisting of citric acid, sodium citrate, sodium chloride, and combinations thereof.

10. The oral composition of claim 1, further comprising a flavor agent, wherein the flavor agent is present in an amount which is effective to abate or eliminate the bitter taste ordinarily imparted by the bitter-tasting agent.

11. An oral composition comprising a bitter-tasting agent, an effective amount of a hydrogenated ethoxylated glycerol ester having an ethoxylation number of 40, an effective amount of mono-ammonium glycyrrhizinate, and an effective amount of sodium citrate, wherein the effective amounts of the hydrogenated ethoxylated glycerol ester, the mono-ammonium glycyrrhizinate and the sodium citrate are effective to mask the bitter taste ordinarily imparted by the bitter-tasting agent.

12. The oral composition of claim 11, wherein the hydrogenated ethoxylated glycerol ester is present in an amount of from about 2.0% to about 4.0% of the total weight of the oral composition, the mono-ammonium glycyrrhizinate is present in an amount of about 0.03% of the total weight of the oral composition, the sodium citrate is present in an amount of about 1.0% of the total weight of the oral composition, and wherein the bitter-tasting agent includes dextromethorphan, pseudoephedrine and guaifenesin.

13. The oral composition of claim 12, further comprising a flavor agent including a cherry flavor, wherein the flavor agent is present in an amount of about 2.0% of the total weight of the oral composition.

14. An oral composition comprising a bitter-tasting agent, an effective amount of a hydrogenated ethoxylated glycerol ester having an ethoxylation number of 40, an effective amount of a sweetener including mono-ammonium glycyrrhizinate and sucralose, and an effective amount of sodium chloride, wherein the effective amounts of the hydrogenated ethoxylated glycerol ester, the sweetener and the sodium chloride are effective to mask the bitter taste ordinarily imparted by the bitter-tasting agent.

15. The oral composition of claim 14, wherein the hydrogenated ethoxylated glycerol ester is present in an amount of about 2.0% of the total weight of the oral composition, the mono-ammonium glycyrrhizinate is present in an amount of about 0.3% of the total weight of the oral composition, the sucralose is present in an amount of about 0.6% of the total weight of the oral composition, the sodium chloride is present in an amount of about 8.0% of the total weight of the oral composition, and wherein the bitter-tasting agent includes atorvastatin.

16. The oral composition of claim 15, further comprising a flavor agent including a citrus mint flavor and a menthol flavor, wherein the flavor agent is present in an amount of about 10.0% of the total weight of the oral composition.

17. An oral composition comprising a bitter-tasting agent, an effective amount of a hydrogenated ethoxylated glycerol ester having an ethoxylation number of 40, an effective amount of sucralose, and an effective amount of citric acid, wherein the effective amounts of the hydrogenated ethoxylated glycerol ester, the sucralose and the citric acid are effective to mask the bitter taste ordinarily imparted by the bitter-tasting agent.

18. The oral composition of claim 17, wherein the hydrogenated ethoxylated glycerol ester is present in an amount of about 2.0% of the total weight of the oral composition, the sucralose is present in an amount of about 2.5% of the total weight of the oral composition, the citric acid is present in an amount of about 0.8% of the total weight of the oral composition, and wherein the bitter-tasting agent includes caffeine.

19. The oral composition of claim 18, further comprising a flavor agent including a coffee flavor, wherein the flavor agent is present in an amount of about 11.0% of the total weight of the oral composition.

20. A method of masking the bitter taste of a bitter-tasting agent in an oral composition comprising the step of adding to the oral composition containing the bitter-tasting agent an effective amount of a taste-receptor blocker, an effective amount of a taste-receptor competitor, and an effective amount of a sweetener.

21. The method of claim 20, wherein the taste-receptor blocker is selected from the group consisting of hydrogenated ethoxylated glycerol esters and combinations thereof, the taste-receptor competitor is selected from the group consisting of citric acid, sodium citrate, sodium chloride, and combinations thereof, and the sweetener is selected from the group consisting of mono-ammonium glycyrrhizinate, sucralose and combinations thereof.

22. The method of claim 21, wherein the bitter-tasting agent is selected from the group consisting of dextromethorphan, chlorhexidine, guaifenesin, pseudoephedrine, caffeine, peroxide, atorvastatin, aspirin, acetaminophen, diphenhydramine, doxylamine, sildenafil citrate, loperamide, and combinations thereof.

23. The method of claim 20, further comprising the step of adding to the oral composition containing the bitter-tasting agent an effective amount of a flavor agent.
